(19) United States
(12) Patent Application Publication
(10) Pub. No.: US 2005/0175566 A1
(43) Pub. Date: Aug. 11, 2005

(54) Cleansing and rinse solutions for skin and hair and method of using

(76) Inventor: Vinit Chantalat, Los Altos Hills, CA (US)

Correspondence Address: CENTRAL COAST PATENT AGENCY, PO BOX 187, AROMAS, CA, 95004, US

(21) Appl. No.: 11/046,588

(22) Filed: Jan. 28, 2005

Related U.S. Application Data
(63) Continuation-in-part of application No. 10/838,776, filed on May 3, 2004.
Application No. 10/838,776 is a continuation-in-part of application No. 10/802,896, filed on Mar. 16, 2004.
Application No. 10/802,896 is a continuation-in-part of application No. 10/697,061, filed on Oct. 29, 2003, now Pat. No. 6,832,634.

(51) Int. Cl. A61K 7/06

(52) U.S. Cl. 424/70.1

(57) ABSTRACT

Methods for cleansing human skin, wounds, and foodstuffs use one or more of a special lotion, baking soda in solution with water, and strong carbonated water. Many diverse applications are taught for treating infections, skin conditions and the like. Further, special apparatus for preparing carbonated water and using same for treatment in many different ways is taught, as are unique dispensing apparatus and methods.

### CROSS-REFERENCE TO RELATED DOCUMENTS

The present application is a continuation-in-part (CIP) of copending application U.S. Ser. No. 10/838,776, filed May 3, 2004. The entire disclosure of the prior application is incorporated into the present application at least by reference.

### FIELD OF THE INVENTION

The present invention is in the field of cleansing generally and has particular application to cleansing foodstuffs, personal hygiene, and cosmetology.

### BACKGROUND OF THE INVENTION

Most people are concerned periodically with having effective methods and materials for cleaning, both things around themselves and their own bodies. It is well-known that poor cleaning procedures and habits can lead to illness.

In the area of personal hygiene, an ever-expanding industry exists in the field of cosmetology for the simple purpose of skin maintenance. This industry is predicated upon the desire of people to be clean, both for an emotional benefit and to stay healthy, and also to attain youthful, shining, and unblemished skin, that is, for the sake of personal appearance. To accomplish these things, many people use facial masks, potentially dangerous acne medication, alcohol, creams, oils, and many other materials and methods that might be detrimental to the skin and to the health of the individual. Also, many of these products and methods are not effective for the purpose of treating underlying causes of acne, skin blemishes and melasma. Some of these products, while somewhat effective to varying degrees, can be unnecessarily harsh and abrasive on the skin. Also, many of these products treat the symptoms of these facial maladies rather than the underlying causes. The underlying causes of these facial maladies are chiefly the following: soot, dead skin cells, bacteria, and residual soap.

One way soot, dead skin cells, bacteria, and residual soap can cause skin blemishes is by clogging skin pores. When skin pores become clogged by such as dead skin cells or soot, bacteria are allowed to propagate under favorable conditions within the skin pores. In turn, pimples may be caused as the body seeks to attack the bacteria in the clogged skin pores.

Another common skin malady is allergic reaction of the skin to soap residue. Soap residue along with some fragrance compounds used in soap may produce an allergic reaction in the skin. The effect of this allergic reaction may manifest itself as either pimples or an unsightly rash.

Soap and water are commonly and conventionally used, typically with mild abrasion, like use of a washcloth, to remove oil, soot, bacteria and dead skin cells. Unfortunately, emulsive effects of soaps are not always strong enough to overcome the adhesive forces between the skin and the oil, soot, bacteria, and dead skin cells. Therefore, a significant amount of oil, soot, bacteria and dead skin cells may maintain a residual presence on the skin or hair.

In order to effectively clean human skin and treat the most common skin maladies, it is necessary to remove bacteria, soot, dead skin cells, oil, and soap residue from the skin. This removal process is not performed effectively by traditional methods and cleansing solutions. What is clearly needed is a method with accompanying cleansing solutions to very effectively remove these residues in cleansing the human body.

In addition to the above it is desirable, especially in the area of residuals such as soot and dirt, but especially bacteria, to have more effective ways of cleaning fruits, vegetables, and other foodstuffs.

### SUMMARY OF THE INVENTION

In a preferred embodiment of the present invention a method for cleansing human skin or hair is provided comprising steps of (a) washing and rinsing the skin or hair, and (b) final rinsing the skin or hair with carbonated water.

In some embodiments the carbonated water is plain carbonated water as sold in markets under the name of soda. In some other embodiments the soda is dispensed from a soda pop dispenser. Also in some embodiments the soda is made in a soda making machine. In still other embodiments the soda is made in a carbonated refreshener shaker. In still further embodiments the carbonated water is plain carbonated water of 8 to 9 volumes gas to water. Further yet the he carbonated water solution may comprise baking soda in solution at from 0.5 to 10% by weight to weight of the water. And further still the carbonated water may further comprise one or more of acetic acid, sodium palmitate, ethanol, a pH corrective solution, lemon juice, or cider vinegar.

In some embodiments the cleansing and rinsing may be for treating a germinal skin infection, a bacterial infection, a fungal infection, or a viral infection. In some cases the carbonated water may further comprise one or both of a fragrance or coloring.

In another aspect of the invention a method for cleansing skin is provided comprising steps of (a) applying a lotion comprising ethanol and acetic acid; and (b) rinsing after step (a) with a solution of baking soda in water.

In some embodiments of the method the ethanol is from 5 to 20% of the water component, and the acetic acid is from 5 to 20% of the water component. Also in some embodiments the lotion further comprises protein at 5-20% of the water component. In some embodiments there may further be one or both of a fragrance and coloring. In still further embodiments the baking soda solution may be from 5 to 20% by weight in water. In still further embodiments the baking soda solution may be further carbonated to 8 to 9 volumes carbon dioxide gas.

In some embodiments the cleansing may be motivated by unpleasant odor, which may be a genital region odor, an armpit odor, or a foot odor. Also in some embodiments the cleansing may be motivated by a skin disorder, which may be an allergic disorder or an age disorder. In still other embodiments the cleansing may be motivated by a skin infection, which may be a bacterial infection, a fungal infection, or a viral infection.

In another aspect of the invention a method for treating cuts and wounds is provided comprising (a) rinsing the cut or wound with a solution of carbonated water; (b) allowing the cut or wound to air dry; and (c) applying a bandage over the cut or wound. In some embodiments the carbonated water has a strength of 8 to 9 volumes of gas in a volume of water at standard temperature and pressure.

In some embodiments the cleansing may be motivated by unpleasant odor, which may be a genital region odor, an armpit odor, or a foot odor. Also in some embodiments the cleansing may be motivated by a skin disorder, which may be an allergic disorder or an age disorder. In still other embodiments the cleansing may be motivated by a skin infection, which may be a bacterial infection, a fungal infection, or a viral infection.

In some embodiments the carbonated water may be plain carbonated water as sold in markets under the name of soda. In others the soda may be dispensed from a soda pop dispenser. In still others the soda may be made in a soda making machine. In still others the soda may be made in a carbonated refreshener shaker.

In yet another aspect of the invention a method for cleaning meat, fish fruit, vegetables, or other food stuffs is provided, comprising (a) cleaning the meat, fish fruit, vegetables, or other food stuffs with plain water; and (b) rinsing the meat, fish fruit, vegetables, or other food stuffs with a solution of carbonated water. In some embodiments of this method the carbonated water has a strength of 8 to 9 volumes of gas in a volume of water at standard temperature and pressure. In some embodiments the carbonated water may be plain carbonated water as sold in markets under the name of soda. In others the soda may be dispensed from a soda pop dispenser. In still others the soda may be made in a soda making machine. In still others the soda may be made in a carbonated refreshener shaker.

In yet another aspect of the invention a lotion for treating skin in a cleansing process is provided, comprising ethanol, acetic acid and water. In some embodiments of the lotion the ethanol and acetic acid each comprise from 5% to 20% by weight of water in the solution. In other embodiments there may be one or both of a fragrance and a coloring present. In another embodiment there may be protein from 0.5 to 20% by weight of water in the solution, and in this case there may also be components for fragrance and coloring.

In another aspect a high strength solution for cleansing and treating human skin is provided, comprising carbonated water and baking soda in solution with the carbonated water. In some embodiments the carbonated water has a strength of 8 to 9 volumes of gas in one volume of water at standard temperature and pressure. In other the baking soda comprises 5 to 20% by weight of baking soda in the water. This solution may be used as ear drops for treating the human outer ear. Such treatment may be for an ear infection or for wax impaction.

In yet another aspect of the invention a system for carbonating water in a tub is provided, comprising a submersible pump having an inlet and a discharge, an external source of pressurized carbon dioxide gas, and a conduit from the external source of gas, the conduit opening at the inlet to the submersible pump. Gas from the external source is injected into water at the inlet to the pump, the gas mixing with the water in the pump, and carbonated water is discharged back into the tub at the pump discharge.

In some embodiments the system further comprises a pressure regulator and flow controller in the conduit between the external source of gas and the inlet to the pump. Also in some embodiments the system further comprises an enclosure for supporting at least the submersible pump and the conduit in the tub. The system may be used for human bathing, for bathing or soaking vegetables or other food stuff, or for washing or rinsing clothing.

In yet another aspect of the invention a method for carbonating water in a tub is provided, comprising placing a submersible pump having an inlet and a discharge in water in the tub, providing a flow of carbon dioxide gas from an external source to an inlet of the pump, and running the pump and injecting carbon dioxide gas at the pump inlet, causing the gas to be mixed with the pumped water ejected at the pump discharge.

In some embodiments there is additionally a step for controlling pressure and flow of the carbon dioxide gas. Also in some embodiments there is a step supporting the pump and the conduit by an enclosure in the tub. In still other embodiments there is a step for bathing a human body in the tub. In still others a step for bathing vegetables or other foodstuff in the tub. In yet others there is a step for washing or rinsing clothing in the tub.

In yet another aspect of the invention a system for carbonating water in a tub is provided, comprising a pump exterior to the tub, having a suction inlet through a first opening in the tub and a discharge conduit to a second opening in the tub, at a point higher than the first opening, a source of pressurized carbon dioxide gas, and a conduit from a source of carbon dioxide gas, the conduit connected to the suction inlet of the pump. Gas from the source is injected into water at the inlet to the pump, the gas mixing with the water in the pump, and carbonated water is discharged back into the tub at the second opening.

In some embodiments there may be a pressure regulator and flow controller in the conduit between the external source of gas and the inlet to the pump. The system may be used for human bathing, for bathing or soaking vegetables or other food stuff, or for washing or rinsing clothing.

In still another aspect of the invention a method for carbonating water in a tub is provided, comprising placing a pump near the tub, the pump having a suction inlet from a first opening in the tub and a discharge line through second opening in the tub, higher than the first opening, providing a flow of carbon dioxide gas from a source to the suction inlet of the pump, and running the pump and injecting carbon dioxide gas at the pump inlet, causing the gas to be mixed with the pumped water ejected at the pump discharge.

In some embodiments of the method there may be a step or controlling pressure and flow of the carbon dioxide gas. There may also be a step for supporting the pump and the conduit by an enclosure in the tub. There may also be a step for bathing a human body in the tub, a step for bathing vegetables or other foodstuff in the tub, or a step for washing or rinsing clothing in the tub.

In yet another embodiment of the invention a dispenser for dispensing carbonated refreshener is provided, comprising a sealable threaded engagement interface for attaching the dispenser to a compatibly threaded neck of a bottle, a user-operable valve for opening and closing a passage from the bottle to an outlet of the dispenser, and an adjustable element for constricting flow of the carbonated refreshener when the valve is open. In some embodiments the user operable valve may comprise a spring-loaded-closed element operated by a cantilevered handle. Also in some embodiments there may be an outlet opening enabled to accept interchangeable nozzles for dispensing carbonated refreshener. In still other embodiments there may be a spout extension enabled to engage the outlet opening, the spout extension for dispensing into a user's hand for face or hand wash or rinse. In yet other embodiments there may be a spout extension for vagina rinse, the extension comprising a reflow channel for relieving pressure in the vagina during dispensing operation.

### BRIEF DESCRIPTION OF THE DRAWING FIGURES

FIG. 1a is a table showing components of a cleansing solution in one embodiment of the present invention.

FIG. 1b is a table showing components of a cleansing solution in an alternative embodiment of the present invention.

FIG. 2a is a flow diagram illustrating one method for cleansing skin according to an embodiment of the present invention.

FIG. 2b is a flow diagram illustrating another method for cleansing skin according to an embodiment of the present invention.

FIG. 2c is a flow diagram illustrating yet another method for cleansing skin according to an embodiment of the present invention.

FIG. 3a is a flow diagram for an exemplary method by which baking soda solution is made for use in a process shown in FIG. 2b.

FIG. 3b is a flow diagram for a process of making carbonated water for use in a process such as shown in FIG. 2a.

FIG. 3c is a flow diagram for a process by which carbonated baking soda solution is made for use in a process such as that shown in FIG. 2c.

FIG. 4 is a chart of a range of volumes of carbon dioxide in water given specific temperature and pressure in a solution in an embodiment of the invention.

FIG. 5 is a flow diagram illustrating a method for cleansing hair in an embodiment of the present invention.

FIG. 6 illustrates a flow diagram with steps for treating a cut or other wound with carbonated water in an embodiment of the invention.

FIG. 7 is a flow diagram with steps for using carbonated water for preparing fruits, vegetables, meat and seafood for packaging in an embodiment of the present invention.

FIG. 8 is an elevation view, partially in cross-section and broken section, illustrating a system for carbonating water in a bath, in an embodiment of the present invention.

FIG. 9 is an elevation view, partially in cross-section and broken section, illustrating an alternative system for carbonating water in a bath, in an embodiment of the present invention.

FIG. 10 is an elevation view, partially in cross-section and broken section of a dispenser for dispensing carbonated refreshener in an embodiment of the present invention.

FIG. 11a is a view of a dispensing spout for hand and face rinse in an embodiment of the present invention.

FIG. 11b is a view of a dispensing spout for vagina rinsing in an embodiment of the present invention.

FIG. 11c is an end view of the dispensing spout of FIG. 11b.

### DESCRIPTION OF PREFERRED EMBODIMENTS

General Description and Teaching

For purposes of description in disclosure of the present invention in various embodiments a carbonated refreshener is a liquid mixed with carbonic gas until fizzy or sparkling. Soda (carbonated water) is plain water mixed with carbonic gas. The chemistry name is Carbonic Acid (H₂CO₃).

Carbonic gas in chemistry terms is Carbon dioxide (CO₂), and is derived from combustion of an organic substance such as charcoal, wood, oil, food, and so on. Exhalations of humans and animals also contain a lot of carbonic gas. Trees release carbonic gas through their leaves at night. Yeast releases carbonic gas while digesting sugar into alcohol. Carbonic gas is an inert gas, non-combustible, and is useful in fire extinguishing, particularly a fire from electrical short circuit.

In various embodiments of the present invention soda (carbonated water) also called Carbonated Refreshener is made in a number of ways. Generally Soda is made by forcing water to mix with carbonic gas, or vice versa, then keeping the soda in a sealed container to avoid pressure leakage. Technical term of forcing is to give kinetic energy to mixing.

In an industrial soda factory water is sprayed into a tank filled with carbonic gas or so-called “Carbonator”. The sprayed water, then, easily mixes with carbonic gas to form soda. In a system in the present invention for making Carbonated Refreshener a bottle is filled with liquid and carbonic gas under pressure, then shaken to force the liquid to mix with carbonic gas at suitable proportion to meet qualification of Carbonated Refreshener, taught later in this specification. This apparatus is called by the inventor a “Carbonated Refreshener Shaker”.

Soda is mildly acidic, which changes the color of Litmus Paper (A substance used to test Acidity or Alkalinity of Solvent) to pink. It has a mild sour taste and is unstable. It can split itself into carbonic gas and water easily. Carbonic gas will then evaporate and the remaining is plain water.

Soda is clear in color, the same as plain water. Under ordinary atmosphere, we can see bubbles floating up from bottom of a glass of soda water. The bubbles are carbonic gas separating from water and evaporating into the atmosphere. Soda has potential energy in itself; that is, if it is enclosed in a container, it will create pressure within the container. Carbonic gas that is separated from water in a sealed container cannot evaporate from the sealed container, therefore pressure is created inside the container. For example, if dirt is added to the soda, the dirt will float up and down, because the carbonic gas that separates from water has enough energy to move the dirt.

The amount of carbonic gas that can mix with water will depend on water temperature and carbonic gas pressure, i.e. at low water temperature, more carbonic gas can combine with water; or at high carbonic gas pressure, more gas can combine with water. The fizziness or sparklingness of soda is measured in “volume”—proportion of carbonic gas per unit of water. For example 5 volume means the volume of carbonic gas at standard temperature and pressure is 5 times the volume of water. Therefore, at a specific pressure and specific temperature of soda, the carbonic gas and water can be combined to a maximum at a specific volume.

A good soda-making machine is one that can efficiently force water and carbonic gas to combine the highest volume of soda at specific pressure and temperature. For example, efficiency of the machine is 50% means that the machine can produce soda with sparklingness only 50% of maximum volume at the pressure and temperature of that machine.

In the priority application Ser. No. 10/697,061 a carbonator according to embodiments of the present invention is described that the inventor calls a CARBOFIZZ Carbonated Refreshener Shaker. This carbonator is able to mix carbonic gas with any kind of liquid, and the level of carbonic gas to be mixed with the liquid may also be specified as follows:

| | | | |
|---|---|---|---|
| Carbonated Drink Refreshener | Fizzy | 3-4 | Volumes |
| Carbonated Cleanser Refreshener | Fizzy | 6-9 | Volumes |
| Carbonated Sprinkle Refreshener | Fizzy | 8-9 | Volumes |
| Carbonated Antiseptic Refreshener | Fizzy | 8-9 | Volumes |

Carbonated Refreshener is mildly acidic, which has no chemical reaction to the human body which also has mild acidity. Human body tissue consists of fat (fatty acid), protein (amino acid), and some liquids (i.e. uric acid, gastric Acid, etc). Carbonated Refreshener has potential energy which is always ready to change into kinetic energy. Because of the instability of Carbonated Refreshener, it is ready to split and discharge carbonic gas at all times. When Carbonated Refreshener touches human skin, the separated carbonic gas will press the skin like a massage. The skin will feel warm around the massaged area. Then, carbonic gas will evaporate into the atmosphere. Similarly, when consuming Carbonated Refreshener, the separated carbonic gas will be absorbed by the small intestine passing through arteries to the lungs and eventually, exhaled to atmosphere.

As a body cleanser Carbonated Refreshener helps eliminate soap residue (Residual Alkalinity) as follows: Soap is a metallic salt of fatty acid derived from mixing alkaline and fat. Soap has mild alkalinity, but human body has mild acidity, therefore soap has a good reaction to body waste by washing the waste from skin. When rinsing out, the soap may leave some residual alkalinity. This residual can react with skin tissue and turn it to brown. In some cases, the soap residue can cause allergic reaction on the skin. Therefore, by using Carbonated Refreshener, which is a mild acid, to wash off alkalinity of soap, the soap residue will become neutralized and rinse away with remaining Carbonated Refresher. Hence, Carbonated Refreshener as a final rinse is excellent in eliminating soap residue. Facial Melanin, Melasma, and skin allergy will be reduced.

Carbonated Refreshener can also push out dust and soot from body skin. Dust and Soot are very fine particles, which can grasp the skin and sink into small pores. And because of its insolubility, dust and soot cannot be easily rinsed out by water, but Carbonated Refreshener can remove the dust and soot. Potential energy from Carbonated Refreshener stimulates movement of dust and soot to loosen them from pores, especially small facial pores. Then the face will feel free and body waste can flow out naturally without clogging the pores. The result is a reduction of facial acne, pimples and rash. Since Carbonated Refreshener helps eliminate dust and soot from clogging pores, the face will instantly feel fresh and clean.

Carbonated Refreshener also helps wash away germs and bacteria from the body. Germs cling to the skin and feed on waste secreted through skin pores. These wastes typically consist of protein, fat, and hormones. As bacteria digests, it produces waste which has a bad odor along with carbonic gas. Soap, foam, and cleanser can only clean germs and bacteria on the skin surface but not those inside the pores. Carbonated Refreshener can eliminate these germs and bacteria from pores. Potential energy from Carbonated Refreshener stimulates movement of germs and bacteria to loosen them from pores, especially small facial pores. The excess Carbonated Refreshener helps convey these bacteria out from the skin, resulting in clean and skin free from bacteria and unpleasant body odor. The body can stay odorless for up to 5 days. Another advantage is that skin cells can regenerate without foreign bacteria feeding on new cells, which leads to faster recovery from wound and infected sores. Particularly with diabetic feet, a decomposed wound is very difficult to dry and recover. Washing skin or immersing diabetic feet in Carbonated Refreshener will help eliminate bacteria from the skin and feet, resulting in quicker recovery and less odor.

When drinking Carbonated Refreshener as it enters the body, it changes from potential energy into kinetic energy to stimulate food in the stomach. So the food can be digested more easily, resulting in increased food absorption by the body. Further Carbonated Refreshener is a product of carbonic gas, the same kind of gas in human exhalation, which can be considered a body's waste. When drinking Carbonated Refreshener, the nature's waste will wash itself naturally.

Carbonic gas that is mixed with beverages will form carbonic acid. Acidity in these beverages can deter the growth of some bacteria. Since carbonic gas is an inert gas, it will cover the beverage's surface so that aerobic bacteria will have no oxygen for growth and will eventually die. However, some fungus and yeast will grow well in carbonated beverages.

Direct disadvantage may occur if drinking Carbonated Beverage with an empty stomach, because carbonic gas from the beverage will activate the empty stomach. The stomach may react as if it has food and start discharging gastric acid, which may cause heartburn. An indirect disadvantage is that the carbonated beverage will wash away human waste inside the body (nature's waste will wash itself naturally). However, if consumed too much, carbonated beverages may not have any waste left to wash out, then some vital elements may be washed out instead.

Research has found that middle aged women who drink too much carbonated beverages have decreased calcium level in the body. Calcium is a bone component; if the amount of Calcium decreases too low, Osteoporosis may develop.

In some cases baking soda which the inventor terms Bubbly Rinse Powder, or Bubbly Rinse Solution, being carbonated water (soda) may be added to a bottle of clean water. The solution is chilled, then compressed with carbonic gas to be ready for use. The Bubbly Rinse Powder or Solution adds additional carbonic gas to a normal Carbonated Refresher. It will be powerful enough to clean and revitalize the skin.

In an embodiment of the invention described in more detail below the face is washed using ordinary steps. As a final step, Carbonated Refreshener is poured in the palm of the hand and splashed on the face thoroughly. The face is then dried with a towel There is no need to wash out Carbonated Refreshener with tap water because Carbonated Refreshener is cleaner than the tap water. Carbonated Refresher should be used entirely after opening a bottle, because any remaining Refreshener will lose its fizziness in the bottle without a good seal. Further, Carbonated Refreshener should be used while cold, so that it will not lose power when it contacts your palm. The refreshener will release energy to massage the skin, causing a warm feeling on the our face. Cold Carbonated Refreshener will give better feeling.

Ordinary steps of face washing are as follows: (1) splash face 2 times with tap water to wet the face, (2) use soap, foam, or cleanser to clean the face thoroughly. (3) While cleaning with soap or cleanser, the lather will become a murky color because soap will dissolve fat, perspiration, and dead tissues to become an emulsion. (4) Wash out all emulsion from the face with water.

Recommendation (A)

1. Every time that the face is washed with soap, foam, or cleanser, follow with Carbonated Refreshener as a final rinse.

2. Facial wash should be extended to under the chin area to prevent germs that may spread from there.

Recommendation (B)

Carbonated Refreshener may be used as a final rinse for bathing and hair washing.

In an embodiment of the invention the face is washed and dried as usual, then an Absorbent cotton pad soaked with Bubbly Rinse Solution is used to wipe the face thoroughly. At least 50 seconds later the solution is rinsed out with Carbonated Refreshener. Then the face is dried with a towel. If the solution touches the eyes, there will be a little stinging, but this is harmless. The sting comes from vinegar in the solution. The Refreshener will quickly relieve the sting in the eyes.

Note:

1. Bubbly Solution also gives good result when applied on your body.

2. The Refreshener to wash out the solution, which is a mix of Bubbly Powder and plain water, can be either carbonated or non-carbonated.

There are direct advantages to washing the face with Carbonated Refreshener, for example:
1. After 1 use, the face will be fresh, light, soft, smooth, moisturized, and vitalized.
2. After 1 week of use, small acne, blackhead, and rash will disappear or be relieved significantly.
3. After 1 month of use, pimples will disappear or be relieved significantly.
4. After 3 months of use, Melanin and Melasma will fade out significantly.
5. When used with Bubbly Rinse Solution, the results above will be achieved faster.

There are also indirect advantages when treating the face with Carbonated Refreshener, for example:
1. More self-confidence, because the clear skin will be admired by others.
2. The brain to works better and increases efficiency, because body liquid can circulate smoothly through your face due to non-clogging of skin pores.
3. Relieves worries about your face, because after washing with Carbonated Refreshener, there will be no stickiness from waste accumulated on your face.
   Note: People, who like to wash their faces with only water due to fear of cleanser need no longer be afraid because Carbonated Refreshener can eliminate Residual Alkalinity better than water or other cleanser.

There are also advantages to bathing with Carbonated Refreshener, for example:
1. After 1 time of bathing, it will eliminate odor, germ, and stickiness. Skin will be smooth, light, soft and more moisturized.
2. No body odor for more than 5 days.
3. If used frequently, the body will not have rash or itch any more.

In embodiments of the invention, after washing the face and skin with soap or cleanser, one should rinse with water (as much as needed) in order to clear the skin from the slipperiness of soap (residual alkalinity). On damp skin, use 1-2 splashes of Carbonated Refreshener to wash the face and skin as the last wash. The slipperiness on the skin will vanish. It is unnecessary to wash with water again after applying Carbonated Refreshener.

Any soap will create slipperiness, which is due to the alkalinity of the soap. It is very difficult to wash away the slipperiness with water, as a result there will always be residual alkalinity (soap residue) after using soap. The skin will react to the soap residue, gradually creating an acidity state. The reaction is accelerated especially when exposed to heat from the sun. Some people may develop darker skin. Some skin may develop an antitoxin to deter the reaction hence developing rashes or becoming allergic to soap.

Residual emulsion is the murky white soap suds. Soap dissolves fat, perspiration, and dead tissue, mixing with water to become emulsion. Residual emulsion, which does not wash away with water, will dry and clog skin pores. As a result, natural skin secretion cannot flow out from the pores therefore creating Acne and Rash. If there is any startup germ, the Acne or Rash may form an infection.

The inventor is aware that some people never wash their faces with soap—washing with water only. Still, those faces are in good condition. The inventor believes that one should not completely stop using soap, partly because soap can dissolve fat and waste produced by our face and skin, therefore it still cleans our pores better than using water alone.

Typically one repeatedly washes residual Alkalinity by water until slipperiness is mostly gone. However, using 1-2 splashes of Carbonated Refreshener as a final rinse is adequate in getting rid of slipperiness. Even though little water is used to wash away soap in the first step (leaving a lot of soap residue), Carbonated Refreshener will immediately eliminate all slipperiness after the first or second splash.

Residual alkalinity and residual emulsion have an alkalinity state, but Carbonated Refreshener is acidic. Naturally, when the two states mix together, the result is neutralization. Carbonic gas in the Carbonated Refreshener will also accelerate the neutralizing reaction, which means as soon as Carbonated Refreshener contacts the residual alkalinity and residual emulsion, the two residuals will be neutralized instantly.

The carbonated refreshener is both a remedy and a prevention for acne and dandruff. For people who recover from having acne and/or dandruff after using the Carbonated Refreshener for just a few times, this is a remedy. For people who does not have any acne and/or dandruff problems and prefer to use Carbonated Refreshener to keep the problem away, this is a prevention.

The Face and forearm, which are frequently exposed to sunlight, wind, and contact, Carbonated Refreshener should be used every evening. During the day when Carbonated Refreshener is unavailable, water can be used instead, but one should not use any cleanser.

Bathing or showering every evening is recommended. However, for people who do not like to bathe everyday, they can use this new technology by bathing with Carbonated Refreshener once a week, and there still will not be any unpleasant body odor at all.

Washing hair every evening is the best way. However, for people who do not like to wash their hair everyday, they can use this new technology by washing hair with Carbonated Refreshener once a week, and there still will not be any dandruff at all.

For the Armpit and feet unpleasant odor will not occur during the 4 days after using Bubbly Solution. Therefore, you can use Bubbly Solution to prevent any odor.

Bubbly Solution may be used to clean the body once a week which will sufficiently be effective for your skin. It is unnecessary to use everyday, because the body is already protected from the elements by your clothing.

Using carbonated refreshener as a final rinse will help eliminate residual alkalinity and residual emulsion on the face. When there is no soap residue to clog the pores, natural liquid in the body, for example facial oil, can flow out conveniently causing the face to become oily quicker than when not washing with Carbonated refreshener.

Furthermore, Residual Alkalinity and Residual Emulsion in original face washing methods will react with the oil from the skin to form salt of oil in its neutral state: non-slippery, dry and frictional. Salt of oil will be eaten by bacteria in the air or existing startup bacteria on the skin, and easily start more bacteria production. Having a lot of bacteria on your face will cause many disadvantages such as wrinkles and dry skin.

People with oily skin get wrinkles slower than those with dry skin, because natural skin oil will coat and protect the face from the atmosphere and bacteria. Fried food spoils more slowly than steamed or boiled food, because the characteristic of oil is a large molecular chain, which is difficult for bacteria to consume. Therefore, the opportunity for bacteria to grow on oily skin is less than on dry skin. Furthermore, natural skin oil will coat and retain moisture in your skin, so people with oily skin will wrinkle slower than dry skin.

People with dry skin, who wash the face with Carbonated Refreshener as a final rinse; find that the skin will release more natural facial oil, hence, remedy and prevent wrinkles and dryness on its own. Moreover after using Carbonated Refreshener to wash the face as a final rinse eliminates startup bacteria which may cause wrinkles and dryness on the face.

Bubbly Solution is in an acid state. So, after a normal face wash with soap or cleanser, Bubbly Solution can easily wipe away any soap residue and emulsion. When Refreshener is mixed with Bubbly Powder to wash as a final rinse, Bubbly Powder will react with the solution to create more carbonic gas, which accelerates neutralization of soap residue and emulsion. Furthermore the excessive carbonic gas will push out the neutralized residue from the face. When there is no residue left to clog the pores, natural liquid from the body i.e. oil from your face, can conveniently flow out. The result is the face will become oily quicker than before.

One may use one of the following methods to remedy and prevent wrinkles and dry skin
Wipe with Bubbly Solution and wash with water mixed Bubbly Powder.
Or wash with Carbonated Refreshener after ordinary step of washing by soap.
But best method is a combination of both methods, i.e. Wipe with Bubbly solution prior to rinsing with Carbonated Refreshener mixed with Bubbly Powder

Using Carbonated Refreshener to wash a scratch or a wound helps the cut or wound to become bacteria free, so new tissue cells can grow more quickly, because carbonic gas in Carbonated Refreshener changes its own potential energy into kinetic energy to push out bacteria and massage tissue cells. When carbonic gas loses its energy, it will dissolve into the atmosphere. This is comparable to a spring coil that is released from its compressed state; its energy will affect surrounding objects as it bounces before losing energy to a stand still. Therefore, when using Carbonated Refreshener to rinse a wound or scratch, the stinging will only be just a short period. When Carbonic gas has evaporated, the Carbonated Refreshener will no longer be active and will not sting.

After rinsing a wound or scratch with Carbonated Refreshener, the wound will be free from bacteria. If the wound is covered with an adhesive bandage, it will be difficult for bacteria in the atmosphere to enter and feed off the liquid in the wound. In such case, the wound can be rinsed once every 2-3 days, otherwise it should be rinsed once a day.

Carbonated Refreshener is made from pure clean water. The fresh water is compressed with carbonic gas then shaken with a CARBOFIZZ Carbonated Refreshener Shaker until fizziness is up to 8-9 volumes. Every step of production involves only clean material and equipment. Both components of Carbonated Refreshener, i.e. water and carbonic gas, are natural materials, which are also present in human body. Carbonic gas is an inert gas used to preserve beverages. Clean water is pure and has no contamination. Therefore, one can use Carbonated Refreshener in many useful ways as follows:
Used for drinking (Hygienic, Pure)
Used for washing anything (Sterile, Sanitary)
Used for washing germ (Disinfectant, Aseptic, Bactericidal, Germicidal)
Used for mending wounds (Healing Accelerator)

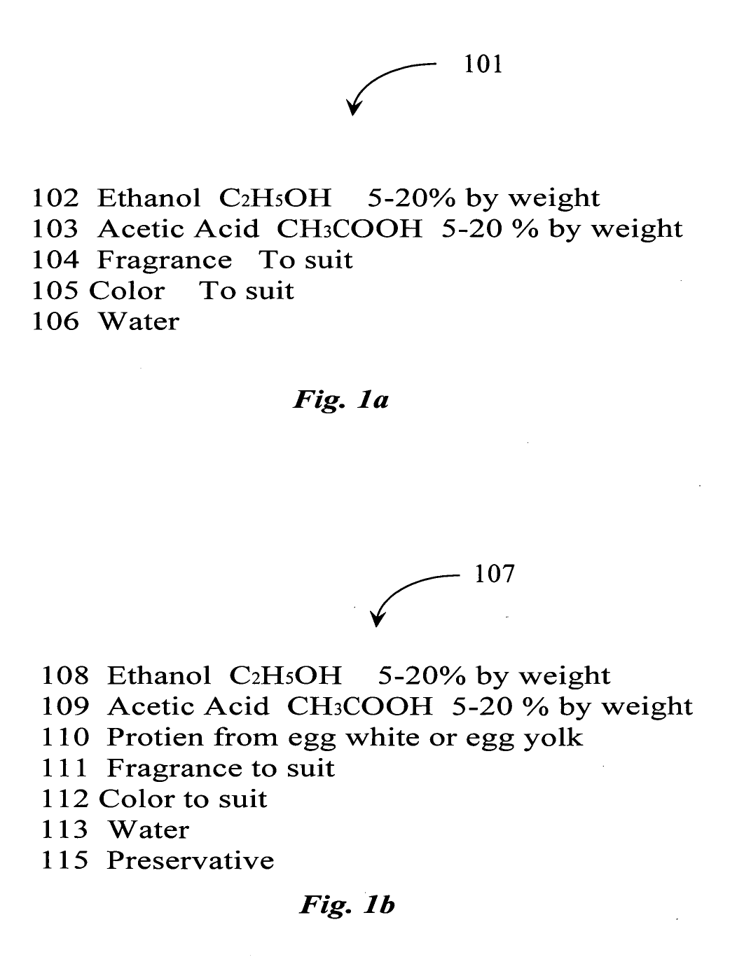

FIG. 1a is a table illustrating components of a unique cleansing solution 101, also termed a lotion by the inventor, according to an embodiment of the present invention. Component 102 is Ethanol, also known as grain alcohol, C₂H₅OH. In one embodiment Ethanol may comprise between 5-20% by weight of cleansing solution 101. Component 102 is Acetic Acid (CH₃COOH), which may also comprise between 5-20% of the weight of cleansing solution 101. A fragrance 104 may also be used and typically has but a very low ratio of weight to total weight. In addition a coloring agent 105 may be present, also typically in very low weight percentage. The balance of the cleansing solution to 100% weight is water 106, preferably distilled water, which is without excessive minerals and the like.

FIG. 1b is a table illustrating an alternative cleansing solution 107 in an alternative embodiment of the present invention. In this alternative element 108 is ethanol, element 109 is acetic acid, and element 110 is protein, which may be derived from egg whites or egg yolks or both. Element 111 is fragrance to suit, element 112 is color to suit, and element 113 is water. In some cases there may also be a preservative 115 to suit.

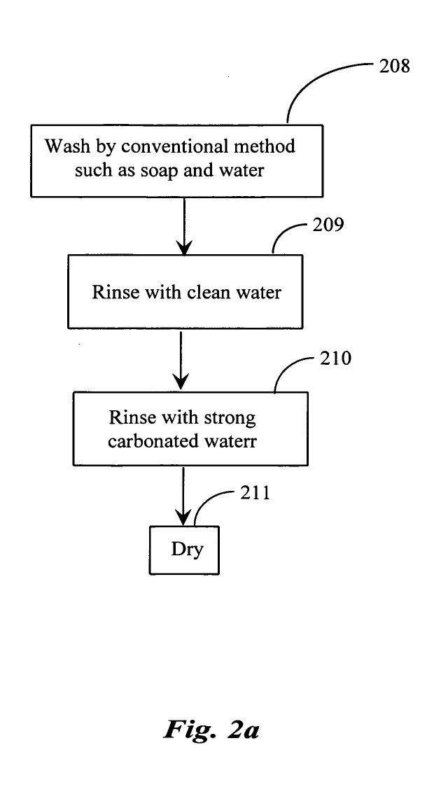

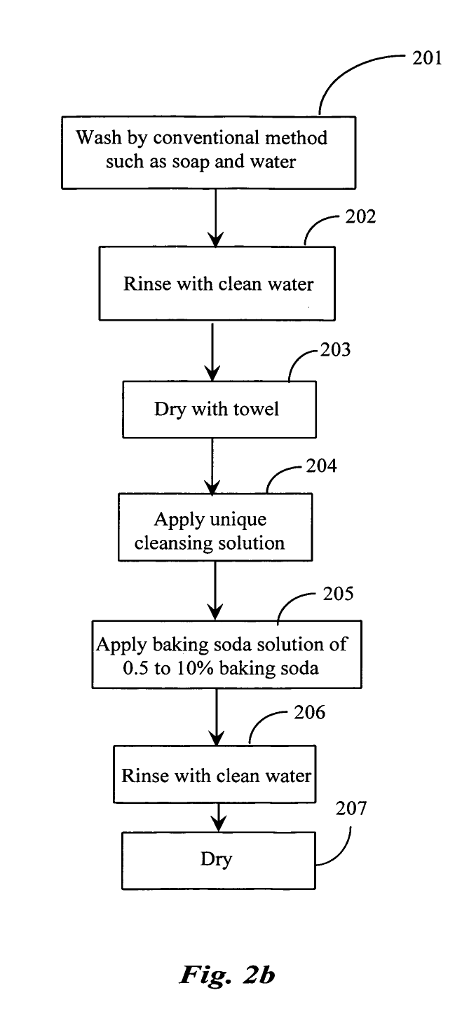

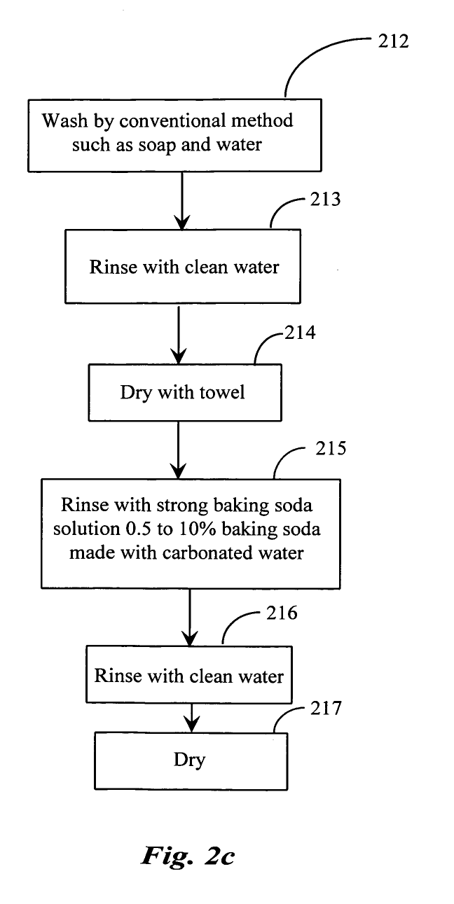

FIG. 2a is a flow diagram showing steps in a new method of cleansing skin according to an embodiment of the present invention. At step 208 a person washes and dries his or her skin using conventional methods and materials, such as soap and water with perhaps mild abrasion. Typically the skin is rinsed after step 208 with warm to hot water in step 209. In step 210 the skin is rinsed with strong carbonated water, and in step 211 the skin is dried.

The method described with the aid of FIG. 2a may be used to treat a variety of skin conditions and problems. For example, the method is believed to be useful for treating skin infections, such as germinal infection, bacterial infection, fungal infections and viral infections.

FIG. 2b is a flow diagram showing steps for cleansing human skin in an alternative method. In the method illustrated in FIG. 2b, in step 201 the skin is washed conventionally by such as using soap and water and perhaps mild abrasion. At step 202 the skin is rinsed with clean water. At step 203 the skin is dried with a towel. At step 204 the cleansing solution as taught in either of FIG. 1a or FIG. 1b is applied. At step 205 a baking soda solution is applied. At step 206 another clean water rinse is accomplished. At step 207 the skin is again dried or allowed to dry.

The method of FIG. 2b is believed to be useful and effective for unpleasant odor, such as a genital region odor, an armpit odor, or a foot odor. Further the method is useful for treating skin disorders, such as an allergic disorder, or an age disorder. It is further useful for treating skin infection, which may be from germinal infection, bacterial infection, fungal infection or viral infection.

FIG. 2c is a flow diagram showing steps for cleansing human skin in yet another alternative method. In this alternative method, at step 212 the skin is washed by a conventional method such as with soap and water. At step 213 the skin is rinsed with clean water. At step 214 the skin is dried, such as with a towel. At step 215 a strong baking soda solution made with carbonated water is used, the solution made with 0.5 to 10 percent baking soda. This particular method is useful in a number of ways, one of which is as drops. The method is useful for such as wax impaction in the outer ear, and also in treating ear infections.

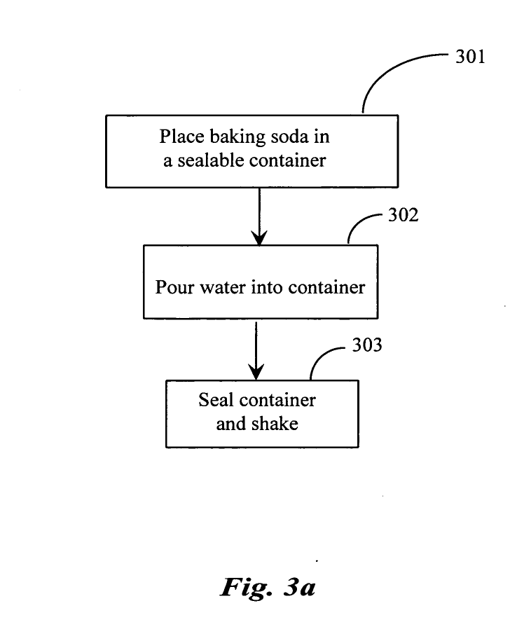

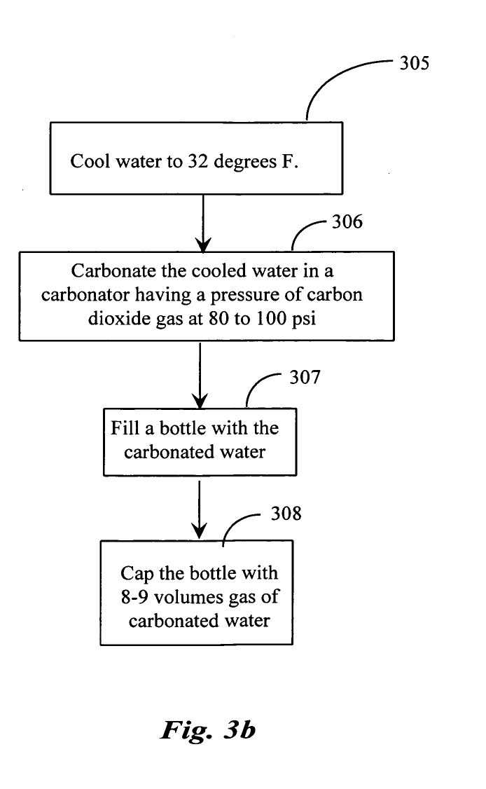

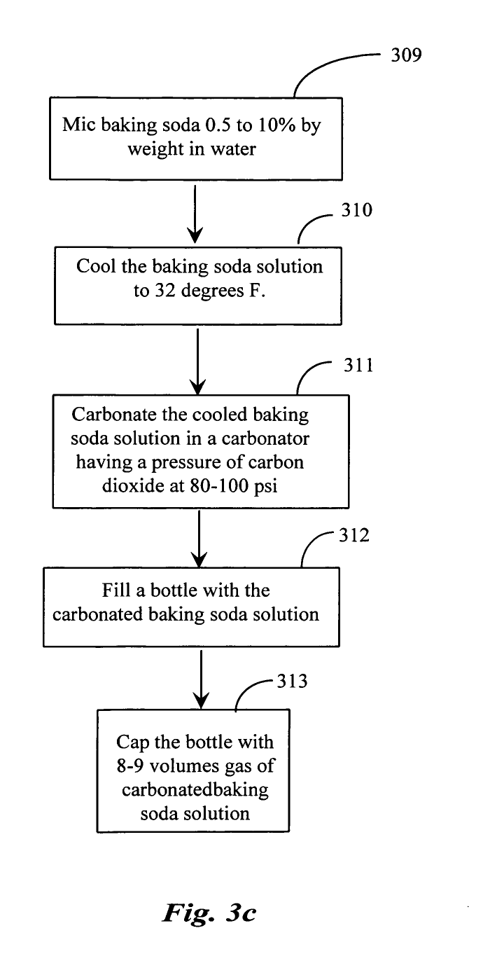

FIG. 3a is a flow diagram showing steps in an exemplary method by which baking soda solution is made for use in various embodiments of the present invention. At step 301 baking soda is placed in a sealable container. It is desirable to have a bottle which holds two liters or more with a screw-on cap. However, other containers may be equally expedient for the production of carbonic water. At step 302 water is poured into the container. At step 303 the container is sealed and shaken. This process produces a baking soda solution useful in embodiments such as that taught with reference to FIG. 2b.

FIG. 3b is a flow diagram illustrating a process for making carbonated water for use in methods according to embodiments of the present invention. At step 305 water is cooled to 32 degrees F. At step 306 the cooled water is carbonated in a carbonator such as one taught in the priority application Ser. No. 10/697,061. At step 307 a bottle is filled with the carbonated water. At step 308 the bottle is capped with 8-9 volumes gas of carbonated water.

FIG. 3c is a flow diagram teaching a process for making carbonated soda water for use in methods according to embodiments of the present invention. At step 309 baking soda at 0.5 to 10% by weight is mixed in water. At step 310 the baking soda solution is cooled to 32 degrees F. At step 311 the baking soda solution is carbonated in a carbonator as described above, and in the priority application Ser. No. 10/697,061, with a pressure in the carbonator of 80 to 100 psi. At step 312 a bottle is filled with the carbonated baking soda solution, and at step 313 the bottle is capped with 8-9 volumes gas of carbonated baking soda solution.

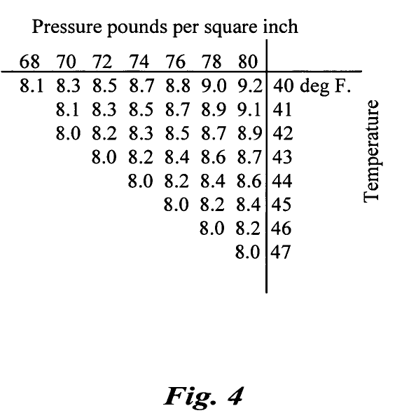

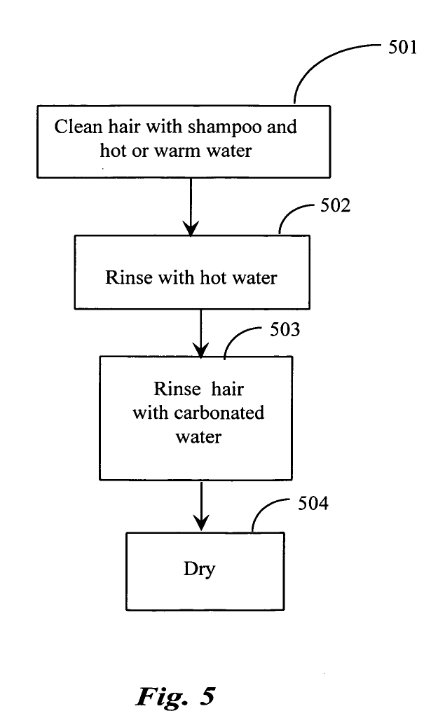

FIG. 4 is a chart of a preferred range of strength of carbon dioxide (CO₂) in water, given a specific range of temperature and a specific range of pressure in the bottle, for effective use in various embodiments of the present invention. The unit indicated at each intersection is the proportion by volume of CO₂ in the solution with the gas at standard temperature and pressure (STP). To understand this chart consider that carbonated water to be measured for strength is poured into a container until the container is substantially full, and then the container is closed. The carbonated water is shaken to develop full pressure. The container is equipped with a temperature probe and a pressure gauge. The temperature and pressure are read, and the intersection of the temperature and pressure are found on the volume chart of FIG. 5. The value at the intersection is the strength of the solution expressed in volumes of gas in the solution at STP. For example, 8.0 read from the chart means that the solution tested has 8 volumes of gas at STP in 1 volume of water. STP is 1 atmosphere pressure and 32 degrees F.

Conversely to the above, if one wishes to create a “5 volume” solution, it is needed to find 5 on the chart and note the temperature and pressure for that strength solution. Then one would cool the water in a carbonator to below the chart temperature and raise the carbon dioxide pressure to above the pressure from the chart; because the carbonator is not 100% efficient.

FIG. 5 is a flow diagram of a method for cleaning hair in an embodiment of the present invention. At step 501 hair is washed with shampoo and hot or warm water. At step 502 the shampoo is rinsed out of the hair with fresh hot or warm water. At step 503 the hair is rinsed with carbonated water made according to an embodiment of the present invention. At step 504 the hair is dried.

Other ingredients may be used with carbonated water in step 503 in some embodiments of the invention. In one embodiment one or more of acetic acid, sodium palmitate or ethanol may be used to augment the cleansing power of the carbonated water. Other additives may be used as well for the purpose of correcting the pH of the hair. In one preferred embodiment, lemon juice may be added. In another embodiment, apple cider vinegar may be added.

Carbonated water at strength preferred in embodiments of the present invention is also useful in applications other than cleansing hair and skin. The high strength carbonated water is also useful for cleaning cuts and wounds, and for cleansing fruits, vegetables, raw meat and fish, as well as other foodstuffs, because the high strength carbonated water has the same loosening and refreshing action in these applications as when used for cleaning the human body or human hair.

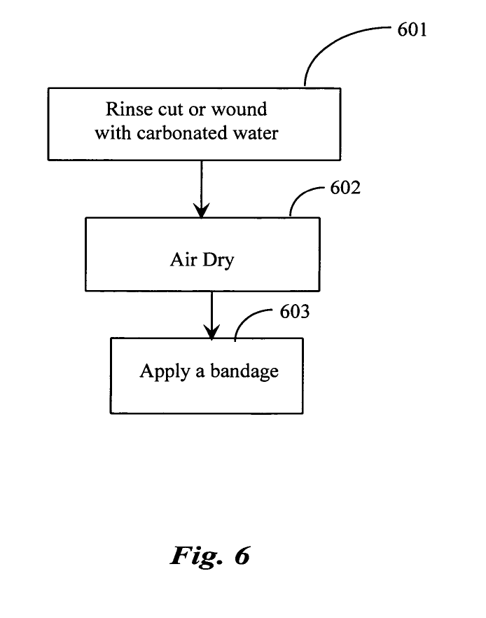

FIG. 6 is a flow chart illustrating a method for treating a cut or a wound with carbonated water. At step 601 the cut or wound is rinsed with carbonated water. At step 602 the cut or wound is allowed to air dry. At step 603 a bandage is applied. The method is effective for situations where the cut or wound is infected by such as, for example, a bacterial infection, a fungal infection, or a viral infection.

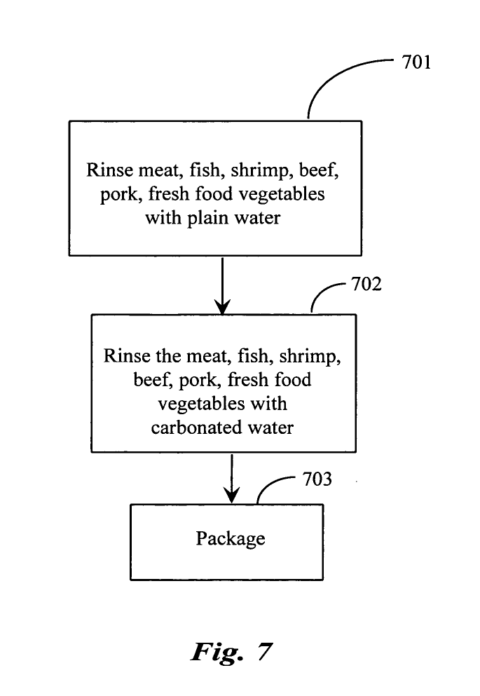

FIG. 7 is a flow diagram with steps for using carbonated water for preparing meat fish shrimp, beef, pork, fresh food vegetables with carbonated water for packaging. At step 701 the items are rinsed with plain water, then at step 702 with carbonated water. At step 703 the items are packaged.

The above teachings comprise in part many methods and benefits using carbonated water for cleansing and treatment of various conditions. In many cases these benefits may be attained by bathing the entire human body in a solution of carbonated water. Bathing with carbonated water may be, for example, a final soak after conventional bathing with soap and water, removing alkali residue, dead skin cells, bacteria, and soot, for example.

In another aspect a full tub of carbonated water may also be useful for a final treatment when washing clothes, to eliminate for example alkali residue, bacteria, dirt and soot. Tubs of carbonated water, produced as described below, using pumps, may also be useful for a final treatment for vegetables and other foodstuff, such as meat and poultry, to rid these items of bacteria dirt, and unwanted chemicals.

The inventor has determined that the use of soaking for a period of time in a solution of 1 1/2 volumes of gas in water may have the same effect as washing or rinsing with a very much stronger solution of 8 to 9 volumes of gas in water. This is because the stronger solution is used for a shorter time, and prolonged immersion in the weaker solution provides more time for the cleansing action.

To take advantage of the benefits of bathing (either people or things) with carbonated water, apparatus and methods are needed to provide sufficient quantity of suitably carbonated water for bathing, and in some cases to maintain the level of carbonation.

In one aspect of the invention a user may prepare a suitable solution for bathing using the apparatus and methods taught above for creating carbonated water of from 8 to 9 volumes of gas. If a tub of water contains, for example, seven gallons of water, and one adds to the tub one gallon of water carbonated to the level of eight volumes, the result will be a tub containing eight gallons at a concentration of about one volume of gas. The inventor believes one to one and one-half volumes of gas is about right for bathing for most purposes; and the skilled artisan may easily measure and prepare the carbonated water in the right concentrations and do the appropriate mixing.

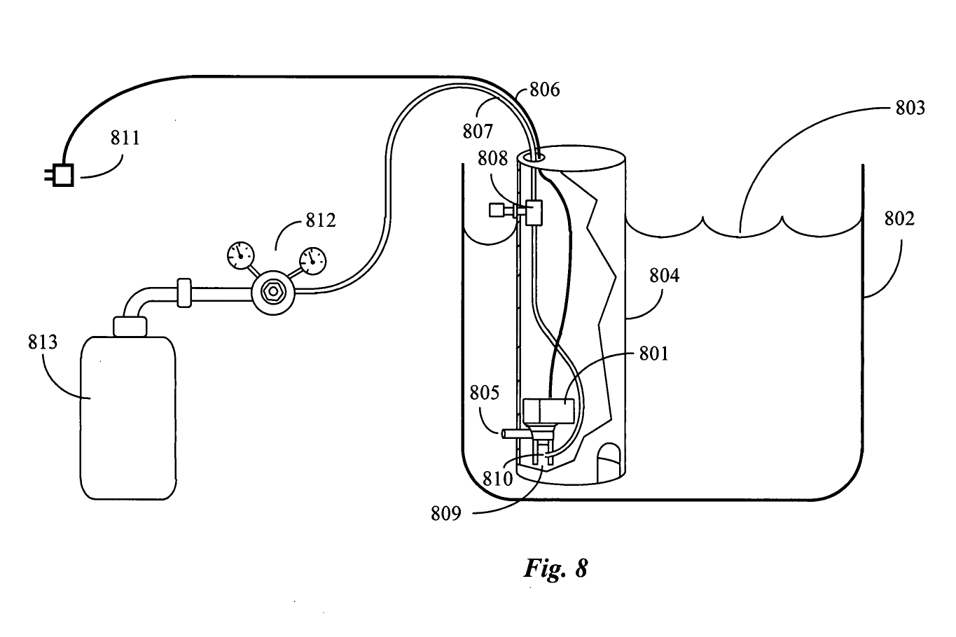

In one embodiment of the invention a submersible pump may be used to provide carbonation to the water in a bath tub. FIG. 8 illustrates such a submersible pump 801, powered through an electrical connection 806 from a plug 811 having an earth ground connection, in a tub 802 containing water 803. In this embodiment carbon dioxide gas from a pressurized supply 813 flows through a pressure regulator 812 and a tubing 807, through a flow regulator 808 the out of a nozzle 810 at the suction inlet 809 of pump 801. The pump and some other apparatus in this embodiment is enclosed in a structure 804 in the tub. Water 803 in tub 802 is drawn into pump 801 through inlet 809, together with injected carbon dioxide gas, and discharged through discharge nozzle 805 back into the tub. Over time the gas content of the water in the tub will decrease by escape of entrained gas at the surface of the water in the tub, and the gradual injection of gas into the pump will keep the content at the desired level. The injection rate is controllable through adjustment of either or both of the pressure of the supply and the regulator 808. Pumps in any case to be used with carbonated water are preferably made of such as stainless steel or plastic, or other relatively non-corrosive material, because the carbonated water can be somewhat corrosive.

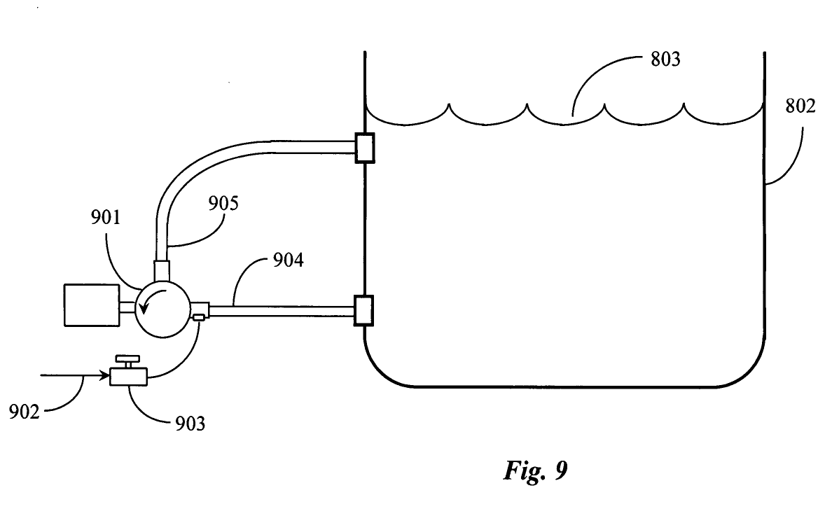

In another embodiment carbonated water for immersion treatment may be produced by a pump exterior to a tub. FIG. 9 is an elevation view of a centrifugal pump 901 mounted outside tub 802, drawing water 803 from the tub via a suction inlet line 904, and discharging back into the tub via a discharge line 905. The suction line is located relatively low in the tub and the discharge line relatively higher in the tub. Carbon dioxide gas from a regulated supply, such as that shown in FIG. 8, is fed into the suction inlet of the pump through a flow regulator 903. Carbonation level in the water is maintained in this case much the same as is done in the embodiment described above with reference to FIG. 8.

In yet another aspect of the present invention carbonated water at various strength may be used therapeutically in a soaking and massage application. By soaking any swollen part of body, such as the foot or the leg, in the carbonated water, swollen parts of body can be gradually healed. The time necessary to have beneficial effect will generally vary inversely to the strength of the carbonated solution. A solution of 8-9 volume for a period of 15 minutes has been found to be effective. For 1-1.5 volume a longer period of time is necessary, say 30 minutes. It is suggested to soak for these periods two times every day. The healing effect comes from the fact that the blood vessels and nerves in the person's skin are massaged by the carbonic gas which gives up potential energy while combining with the water, and then turns to kinetic energy in the form of massaging energy. The user can see the gas becoming small bubbles on the skin and feel warmth on the skin. When the bubbles become bigger they break away, rise to the surface and evaporate to the atmosphere. In some cases the carbonated water may be heated or cooled, and in some cases water circulation, such as jet action, may be used as well, either with or without heat or cold.

The massaging energy aids and enhances blood circulation in the skin, thus gradually relieving swelling symptoms. The causes of swelling symptoms may be, for example, from lack of movement for a long period of time like Economy Syndrome, or may be from an infected wound (bacterial, fungal, viral), or may be from such as a diabetes.

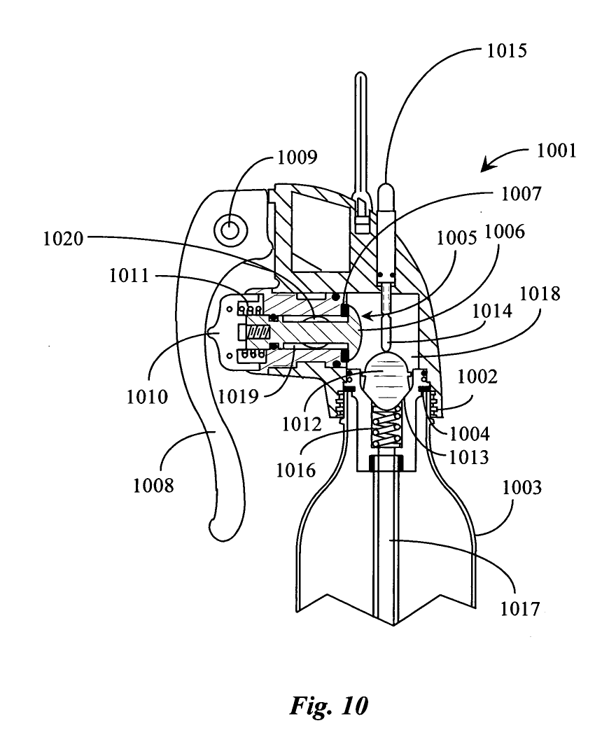

In another aspect of the present invention a hand-operable dispenser is provided in conjunction with a bottle for storing and dispensing carbonated refreshener for various purposes. The carbonated refreshener, as described above, may be simply carbonated water at some appropriate concentration, or may also have other ingredients, such as, for example, a perfume agent acetic acid, sodium palmitate, ethanol, a pH corrective solution, lemon juice, or cider vinegar. FIG. 10 is an elevation cross-section view of a dispenser in one embodiment of the invention. Dispenser 1001 has a threaded interface 1002 for engaging a matching thread on a bottle neck of a bottle 1003, and a seal member 1004 for providing a hermetic seal when engaged.

In this embodiment of the dispenser a spring-loaded valve 1005 has a head 1006 that seats on a seal ring 1007. The valve head may be translated away from seal 1007 by squeezing handle 1008, which is pivoted at shaft 1009 and, when squeezed, depresses actuator 1010 against spring 1011, moving valve 1005. Releasing the handle allows the valve to close again.

Flow of material from bottle 1003 is regulated by a flow cone 1012 which may be maintained at a fixed distance from seat 1013 by adjustment of a threaded screw stem by rotating knob 1015. The flow cone is urged away from seat 1013 by spring 1016.

In operation carbonated refreshener is placed into bottle 1003 and the dispenser 1001 is engaged on the bottle, sealing the bottle and its contents. Pressure will build in the bottle by the natural escape of gas from the refreshener fluid, and the pressurizing can be accelerated by shaking the bottle. A dispenser tube extends to near the bottom of the bottle, so pressure above the fluid in the bottle will tend to urge liquid up the tube, past flow cone 1012 (assuming adjusted open to some degree), and into volume 1018. When a user depresses handle 1008, valve 1005 opens, and fluid will flow from volume 1018 into space 1019 around the valve and out a side opening 1020. Each time the system is used, the pressure will drop, but escape of gas from the fluid over a short time period will increase the gas pressure for the next use.

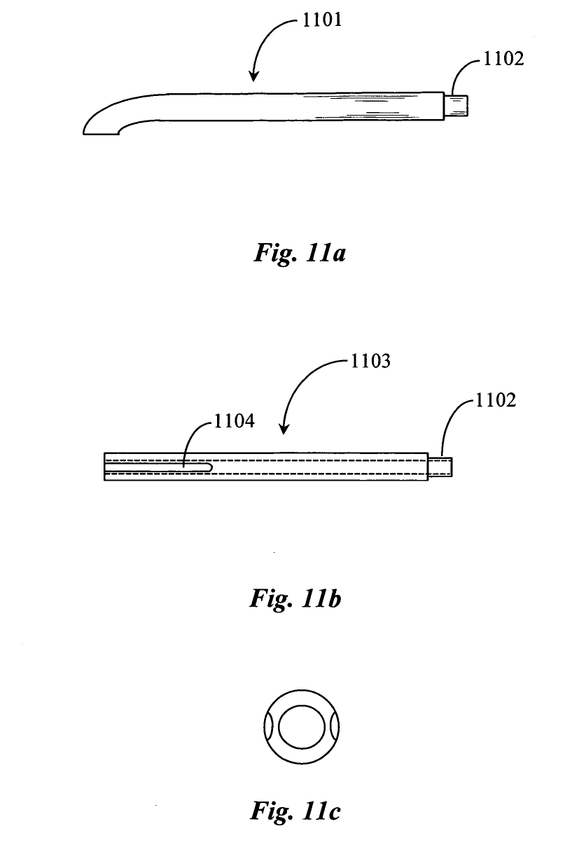

There are a number of uses for the bottle and dispenser, among them dispensing carbonated refreshener for washing or rinsing the face and hands, and also for irrigating other body areas, such as a vagina rinse. For dispensing refreshener for the face and hands a simple tube will do. For irrigating the vagina with carbonated refreshener a tube with an overflow trough is provided. FIG. 11a illustrates a tube to connect at opening 1020 (FIG. 10) for use to dispense refreshener for the face and hands. An engagement interface 1102 is provided to fit snugly into opening 1020 (FIG. 10). The tube may be curved as shown for dispensing fluid. FIG. 11b illustrates a tube for vagina wash, having the same interface 1102 as tube 1101, and also having two reflow troughs 1104, amounting to grooves in the outside surface of the tube as well as a central passage, the reflow troughs allowing fluid to escape from the vagina with the tube inserted, to avoid pressurizing the space inside the vagina, which might injure internal organs.

It will be apparent to the skilled artisan that there are numerous changes that may be made in embodiments described herein without departing from the spirit and scope of the present invention. Many of these changes have already been detailed. Other features not described in this specification, but known to one skilled in the art may also be integrated as well, without departing from the spirit and scope of the present invention. The methods and accompanying solutions of the present invention should therefore be afforded the broadest possible scope under examination. As such, the invention taught herein by specific examples is limited only by the claims that follow.

1. A method for cleansing human skin or hair comprising steps of:
(a) washing and rinsing the skin or hair; and
(b) final rinsing the skin or hair with carbonated water.

2. The method of claim 1 wherein the carbonated water is plain carbonated water as sold in markets under the name of soda.

3. The method of claim 2 wherein the soda is dispensed from a soda pop dispenser.

4. The method of claim 2 wherein the soda is made in a soda making machine.

5. The method of claim 2 wherein the soda is made in a carbonated refreshener shaker.

6. The method of claim 1 wherein the carbonated water is plain carbonated water of 8 to 9 volumes gas to water.

7. The method of claim 6 wherein the carbonated water solution further comprises baking soda in solution at from 0.5 to 10% by weight to weight of the water.

8. The method of claim 1 wherein the carbonated water further comprises one or more of acetic acid, sodium palmitate, ethanol, a pH corrective solution, lemon juice, or cider vinegar.

9. The method of claim 1 wherein the cleansing and rinsing is for treating a germinal skin infection.

10. The method of claim 9 wherein the infection is a bacterial infection.

11. The method of claim 9 wherein the infection is a fungal infection.

12. The method of claim 9 wherein the infection is a viral infection.

13. The method of claim 1 wherein the carbonated water further comprises one or both of a fragrance or coloring.

14. A method for cleansing skin comprising steps of:
(a) applying a lotion comprising ethanol and acetic acid; and
(b) rinsing after step (a) with a solution of baking soda in water.

15. The method of claim 14 wherein the ethanol is from 5 to 20% of the water component, and the acetic acid is from 5 to 20% of the water component.

16. The method of claim 15 wherein the lotion further comprises protein at 5-20% of the water component.

17. The method of claim 15 further comprising one or both of a fragrance and coloring.

18. The method of claim 16 further comprising one or both of a fragrance and coloring.

19. The method of claim 14 wherein the baking soda solution is from 0.5 to 20% by weight in water.

20. The method of claim 19 wherein the baking soda solution is further carbonated to 8 to 9 volumes carbon dioxide gas.

21. The method of claim 14 wherein the cleansing is motivated by unpleasant odor.

22. The method of claim 21 wherein the odor is a genital region odor.

23. The method of claim 21 wherein the odor is an armpit odor.

24. The method of claim 21 wherein the odor is a foot odor.

25. The method of claim 14 wherein the cleansing is motivated by a skin disorder.

26. The method of claim 25 wherein the skin disorder is an allergic disorder.

27. The method of claim 26 wherein the skin disorder is an age disorder.

28. The method of claim 14 wherein the cleansing is motivated by a skin infection.

29. The method of claim 28 wherein the infection is a bacterial infection.

30. The method of claim 28 wherein the infection is a fungal infection.

31. The method of claim 28 wherein the infection is a viral infection.

32. A method for treating cuts and wounds comprising:
(a) rinsing the cut or wound with carbonated water;
(b) allowing the cut or wound to air dry; and
(c) applying a bandage over the cut or wound.

33. The method of claim 32 wherein the solution of carbonated water has a strength of 8 to 9 volumes of gas in a volume of water at standard temperature and pressure.

34. The method of claim 32 wherein the cut or wound is infected.

35. The method of claim 34 wherein the infection is a bacterial infection.

36. The method of claim 34 wherein the infection is a fungal infection.

37. The method of claim 34 wherein the infection is a viral infection.

38. The method of claim 32 wherein the carbonated water is plain carbonated water as sold in markets under the name of soda.

39. The method of claim 38 wherein the soda is dispensed from a soda pop dispenser.

40. The method of claim 38 wherein the soda is made in a soda making machine.

41. The method of claim 38 wherein the soda is made in a carbonated refreshener shaker.

42. A method for cleaning meat, fish fruit, vegetables, or other food stuffs, comprising:
(a) cleaning the meat, fish fruit, vegetables, or other food stuffs with plain water; and
(b) rinsing the meat, fish, fruit, vegetables, or other food stuffs with carbonated water.

43. The method of claim 42 wherein the carbonated water has a strength of 8 to 9 volumes of gas in a volume of water at standard temperature and pressure.

44. The method of claim 42 wherein the carbonated water is plain carbonated water as sold in markets under the name of soda.

45. The method of claim 44 wherein the soda is dispensed from a soda pop dispenser.

46. The method of claim 44 wherein the soda is made in a soda making machine.

47. The method of claim 44 wherein the soda is made in a carbonated refreshener shaker.

48. A lotion for treating skin in a cleansing process, comprising:
Ethanol;
acetic acid; and
water.

49. The lotion of claim 48 wherein the ethanol and acetic acid each comprise from 0.5% to 20% by weight of water in the solution.

50. The lotion of claim 49 further comprising one or both of a fragrance and a coloring.

51. The lotion of claim 48 further comprising protein from 0.5% to 20% by weight of water in the solution.

52. The lotion of claim 49 further comprising one or both of a fragrance and a coloring.

53. A high strength solution for cleansing and treating human skin, comprising:
carbonated water; and
baking soda in solution with the carbonated water.

54. The solution of claim 53 wherein the carbonated water has a strength of 8 to 9 volumes of gas in one volume of water at standard temperature and pressure.

55. The solution of claim 53 wherein the baking soda comprises 0.5 to 20% by weight of baking soda in the water.

56. The high strength solution of claim 53 used as ear drops for treating the human outer ear.

57. The high strength solution of claim 56 wherein the treatment is for an ear infection.

58. The high strength solution of claim 56 wherein the treatment if for wax impaction.

59. A system for carbonating water in a tub, comprising:
a submersible pump having an inlet and a discharge;
an external source of pressurized carbon dioxide gas;
a conduit from the external source of gas, the conduit opening at the inlet to the submersible pump;
wherein gas from the external source is injected into water at the inlet to the pump, the gas mixing with the water in the pump, and carbonated water is discharged back into the tub at the pump discharge.

60. The system of claim 59 further comprising a pressure regulator and flow controller in the conduit between the external source of gas and the inlet to the pump.

61. The system of claim 61 further comprising an enclosure for supporting at least the submersible pump and the conduit in the tub.

62. The system of claim 60 used for human bathing.

63. The system of claim 60 used for bathing or soaking vegetables or other food stuff.

64. The system of claim 60 used for washing or rinsing clothing.

65. A method for carbonating water in a tub, comprising:
placing a submersible pump having an inlet and a discharge in water in the tub;
providing a flow of carbon dioxide gas from an external source to an inlet of the pump; and
running the pump and injecting carbon dioxide gas at the pump inlet, causing the gas to be mixed with the pumped water ejected at the pump discharge.

66. The method of claim 65 including a step for controlling pressure and flow of the carbon dioxide gas.

67. The method of claim 65 including supporting the pump and the conduit by an enclosure in the tub.

68. The method of claim 65 comprising a step for bathing a human body in the tub.

69. The method of claim 65 comprising a step for bathing vegetables or other foodstuff in the tub.

70. The method of claim 65 comprising a step for washing or rinsing clothing in the tub.

71. A system for carbonating water in a tub, comprising:
a pump exterior to the tub, having a suction inlet through a first opening in the tub and a discharge conduit to a second opening in the tub, at a point higher than the first opening;
a source of pressurized carbon dioxide gas; and
a conduit from a source of carbon dioxide gas, the conduit connected to the suction inlet of the pump;
wherein gas from the source is injected into water at the inlet to the pump, the gas mixing with the water in the pump, and carbonated water is discharged back into the tub at the second opening.

72. The system of claim 71 further comprising a pressure regulator and flow controller in the conduit between the external source of gas and the inlet to the pump.

73. The system of claim 71 used for human bathing.

74. The system of claim 71 used for bathing or soaking vegetables or other food stuff.

75. The system of claim 71 used for washing or rinsing clothing.

76. A method for carbonating water in a tub, comprising:
placing a pump near the tub, the pump having a suction inlet from a first opening in the tub and a discharge line through second opening in the tub, higher than the first opening;
providing a flow of carbon dioxide gas from a source to the suction inlet of the pump; and
running the pump and injecting carbon dioxide gas at the pump inlet, causing the gas to be mixed with the pumped water ejected at the pump discharge.

77. The method of claim 76 including a step for controlling pressure and flow of the carbon dioxide gas.

78. The method of claim 76 including supporting the pump and the conduit by an enclosure in the tub.

79. The method of claim 76 comprising a step for bathing a human body in the tub.

80. The method of claim 76 comprising a step for bathing vegetables or other foodstuff in the tub.

81. The method of claim 76 comprising a step for washing or rinsing clothing in the tub.

82. A dispenser for dispensing carbonated refreshener, comprising:
a sealable threaded engagement interface for attaching the dispenser to a compatibly threaded neck of a bottle;
a user-operable valve for opening and closing a passage from the bottle to an outlet of the dispenser; and
an adjustable element for constricting flow of the carbonated refreshener when the valve is open.

83. The dispenser of claim 82 wherein the user operable valve comprises a spring-loaded-closed element operated by a cantilevered handle.

84. The dispenser of claim 82 comprising an outlet opening enabled to accept interchangeable nozzles for dispensing carbonated refreshener.

85. The dispenser of claim 84 further comprising a spout extension enabled to engage the outlet opening, the spout extension for dispensing into a user's hand for face or hand wash or rinse.

86. The dispenser of claim 84 further comprising a spout extension for vagina rinse, the extension comprising a reflow channel for relieving pressure in the vagina during dispensing operation.

87. A method for treating swelling of parts of a human body, comprising the steps of:
(a) preparing a solution of carbonated water; and
(b) immersing the swollen body part in the carbonated water for a period of time.

88. The method of claim 87 wherein the carbonated water is prepared of from 1 to 10 volumes of gas to water.

89. The method of claim 87 wherein the carbonated water is heated or cooled.

90. The method of claim 87 wherein water circulation is used in step (b).
